(19) United States
(12) Patent Application Publication
(10) Pub. No.: US 2011/0000500 A1
(43) Pub. Date: Jan. 6, 2011

(54) DILUTION THICKENING, LIQUID CLEANSING COMPOSITIONS

(76) Inventor: Amauri Zanini Luna, Vinhedo - SP (BR)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/867,298

(22) PCT Filed: Feb. 16, 2009
(86) PCT No.: PCT/EP09/51760

§ 371 Date: Sep. 24, 2010

(30) Foreign Application Priority Data
Feb. 22, 2008 (EP) 08151798.9
Jul. 2, 2008 (EP) 08159488.9

(51) Int. Cl. A45D 44/00; A61Q 5/02; A61Q 19/10; A61K 8/58

(52) U.S. Cl. 132/221; 510/159; 510/125

(57) ABSTRACT

The invention provides a liquid cleansing composition comprising: (i) 1 to 30% by weight, based on the total weight of the composition, of anionic surfactant; (ii) up to 5% by weight, based on the total weight of the composition, of amphoteric surfactant; (iii) up to 2% by weight, based on the total weight of the composition, of electrolyte; (iv) at least 0.5% by weight, based on the total weight of the composition, of monoalkanolamide surfactant, and (v) from 50 to 94.5% by weight, based on the total weight of the composition, of water, wherein the composition has a viscosity upon dilution which is greater than its viscosity prior to dilution.

### FIELD OF THE INVENTION

The present invention relates to liquid cleansing compositions which are suitable for washing of skin or hair, and which have a viscosity which increases during dilution.

### BACKGROUND AND PRIOR ART

The use of salts to thicken surfactant systems and enhance viscosity is described for example in CA 2,211,313. Typically, a so-called peak viscosity is achieved when salt is first added, and further addition of salt leads to viscosity reduction (this is known as oversalting). When the composition is diluted, the “oversalted” composition then increases in viscosity once more in a process referred to as “dilution thickening.”

Dilution thickening is a desirable property for liquid cleansing compositions such as skin or hair washing compositions because it reduces product wastage caused by the liquid product slipping between the fingers or being rinsed away before the consumer has had time to use it. Dilution thickening also improves lather quality and makes product dosage easier to control for the consumer. Dilution thickening also makes spreading of the product onto the skin or hair easier to control for the consumer.

Generally the dilution thickening effect is achieved by using relatively large amounts of salt (e.g. greater than 5% by weight). A problem with using large amounts of salt is that this can lead to a cloudy, hazy or opalescent product, or a product which is prone to phase separation.

U.S. Pat. No. 6,919,303 describes how an associative thickener such as PEG-200 glyceryl tallowate or PEG-7 glyceryl cocoate lessens the amount of salt needed to induce dilution thickening to as low as 2% by weight.

The present inventors have found that the use of a monoalkanolamide surfactant lessens the amount of salt needed to induce dilution thickening to as low as 1% by weight or less.

U.S. Pat. No. 6,919,303 mentions that its compositions may also comprise coconut acyl monoethanolamides as suds boosters, but does not indicate that such materials might play any role in the induction of dilution thickening.

WO 01/40431 describes a dilution-thickening hand soap composition which can include as a viscosity builder an alkanolamide derived from a fatty acid, such as coco diethanolamide, at a level of between 6 wt % and 25 wt %. The application also exemplifies the inclusion of sodium chloride in the concentrate at levels of 7% upwards.

WO 03/014275 describes a dilution-thickening liquid soap. composition based on a particular amine oxide surfactant; specific examples are shown which include both sodium chloride and cocamide diethanolamide at levels of 6% upwards.

US 2005/0043193, WO 95/02664, WO 96/21721, WO 96/32464, US 2007/0287648 and WO 96/03483 all describe other dilution-thickening systems.

### SUMMARY OF THE INVENTION

The invention provides a liquid cleansing composition comprising:
(i) 1 to 30% by weight, based on the total weight of the composition, of anionic surfactant;
(ii) up to 5% by weight, based on the total weight of the composition, of amphoteric surfactant;
(iii) up to 2% by weight, based on the total weight of the composition, of electrolyte;
(iv) at least 0.5% by weight, based on the total weight of the composition, of monoalkanolamide surfactant, and
(v) from 50 to 94.5% by weight, based on the total weight of the composition, of water,

wherein the composition has a viscosity upon dilution which is greater than its viscosity prior to dilution.

The invention also provides the use of monoalkanolamide surfactant for lessening the amount of electrolyte required to induce dilution thickening in a liquid cleansing composition, such as the composition as defined above.

### DETAILED DESCRIPTION OF THE INVENTION

Anionic Surfactant

Compositions according to the invention comprise 1 to 30% by weight, based on the total weight of the composition, of anionic surfactant.

Examples of suitable anionic surfactants are the alkyl sulphates, alkyl ether sulphates, alkaryl sulphonates, alkanoyl isethionates, alkyl succinates, alkyl sulphosuccinates, N-alkyl sarcosinates, alkyl phosphates, alkyl ether phosphates, alkyl ether carboxylates, alkyl ester carboxylates, and alpha-olefin sulphonates, especially their alkali metal, ammonium or alkanolammonium salts. The alkyl and acyl groups generally contain from 8 to 22 carbon atoms, may be straight or branched chain, and may be saturated or unsaturated. The alkyl ether sulphates, alkyl ether phosphates and alkyl ether carboxylates may contain from 1 to 10 ethylene oxide or propylene oxide units per molecule.

A preferred class of anionic surfactants for use in the invention are alkyl ether sulphates of general formula:

R—O—(CH₂—CH₂—O)ₙ—SO⁻₃M⁺

in which R is a straight or branched chain alkyl group having 10 to 18, preferably 12 to 14 carbon atoms, X is a number that represents the average degree of ethoxylation and ranges from 1 to 5, preferably from 2 to 3.5, and M is a alkali metal, ammonium or alkanolammonium cation, preferably sodium, potassium, monoethanolammonium or triethanolammonium, or a mixture thereof.

Mixtures of any of the foregoing anionic surfactants may also be suitable.

The total amount of anionic surfactant in compositions of preferably ranges from 2 to 12%, more preferably from 4 to 10% by weight based on the total weight of the composition.

Amphoteric Surfactant

Compositions according to the invention comprise up to 5% by weight, based on the total weight of the composition, of amphoteric surfactant.

Examples of suitable amphoteric surfactants include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates. The alkyl and acyl groups generally contain from 8 to 22 carbon atoms, may be straight or branched chain, and may be saturated or unsaturated.

A preferred class of amphoteric surfactants for use in the invention are alkyl amidopropyl betaines of general formula:

R—CO—NH—(CH₂)₃—N⁺(CH₃)₂—CH₂COO⁻

in which R is a straight or branched chain alkyl group having 10 to 18, preferably 12 to 14 carbon atoms.

Mixtures of any of the foregoing amphoteric surfactants may also be suitable.

The total amount of amphoteric surfactant in compositions of the invention preferably ranges from 0.1 to 4%, more preferably from 0.5 to 2.5% by weight based on the total weight of the composition.

Electrolyte

Compositions according to the invention comprise up to 2% by weight, based on the total weight of the composition, of electrolyte.

Examples of suitable electrolytes include halides of ammonium, alkanolammonium, alkali metals, alkaline earth metals, and other metals such as aluminium and zinc; sulphates and phosphates of ammonium, alkanolammonium, alkali metals, alkaline earth metals, and other metals such as aluminium and zinc; and alkali metal silicates.

Preferred examples of electrolytes for use in the invention are sodium chloride, potassium chloride, sodium sulphate, potassium sulphate, magnesium chloride, magnesium sulphate, zinc sulphate, ammonium chloride and monoethanolammonium chloride. Sodium chloride is the most preferred electrolyte.

Mixtures of any of the foregoing electrolytes may also be suitable.

The total amount of electrolyte in compositions of the invention preferably ranges from at least 0.1%, conveniently 0.1% to 2.0%, conveniently 0.3 to 1.8%, preferably from 0.3 to 1.5%, more preferably from 0.5 to 1.2%, preferably from 0.5% to 1.1%, most preferably from 0.8 to 1.2%, and in some cases 0.8% to 1% by weight based of the total weight of the composition.

Monoalkanolamide Surfactant

Compositions according to the invention comprise at least 0.5% by weight, based on the total weight of the composition, of monoalkanolamide surfactant.

By “monoalkanolamide surfactant” is generally meant a condensate formed from the reaction between a C₂-C₆ alkanolamine and a fatty acid having from about 8 to about 24 carbon atoms.

Examples of suitable C₂-C₆ alkanolamines used to form the monoalkanolamide surfactant include monoethanolamine, isopropanolamine, diethylene glycolamine (2-(2-aminoethoxy)ethanol), and mixtures thereof. Monoethanolamine is preferred.

Examples of suitable fatty acids used to form the monoalkanolamide surfactant include lauric acid, palmitic acid, stearic acid, oleic acid and linoleic acid; and fatty acids derived from plant oils such as coconut oil, soybean oil, canola oil (genetically modified Canadian rapeseed oil), wheat germ oil, peanut oil, corn oil and olive oil. Mixtures of any of the above fatty acids may also be used.

Preferred monoalkanolamide surfactants for use in the invention include coconut monoethanolamide (cocamide MEA), lauric monoethanolamide, stearic monoethanolamide, oleic monoethanolamide, and linoleic monoethanolamide. Cocamide MEA is particularly preferred.

Mixtures of any of the foregoing monoalkanolamide surfactants may also be suitable.

The total amount of monoalkanolamide surfactant in compositions of the invention preferably ranges from 0.5 to 2.0%, more preferably from 0.6 to 1.2%, most preferably from 0.8 to 1.0% by weight based on the total weight of the composition.

Water

Compositions according to the invention comprise from 50 to 94.5% by weight, based on the total weight of the composition, of water.

Preferably distilled or de-ionised water is used.

The amount of water in compositions of the invention preferably ranges from 60 to 90%, more preferably from 70 to 90%, most preferably from 80 to 90% by weight based on the total weight of the composition.

Optionals

Optionally, the liquid cleansing composition of the invention may contain further ingredients as described below to enhance performance and/or consumer acceptability.

For example, skin or hair care actives may be included to provide skin or hair benefits in addition to cleansing. Examples of such benefits include hydration, nutrition, softness, protection and revitalisation.

Examples of typical skin or hair actives include glycerine, sorbitol, vitamins, botanical extracts, fruit extracts, sugar derivatives, alpha hydroxy acids, isopropyl myristate, UV filters, fatty acids and their esters, silicones, amino acids, hydrolysed proteins, cationic surfactants, essential oils, vegetable oils, mineral oils, sterols, cationic polymers, exfoliating agents and bactericides.

Other optional ingredients include fragrance, dyes and pigments, pH adjusting agents, pearlescers or opacifiers, viscosity modifiers and preservatives.

The above optional ingredients will generally be present individually in an amount ranging from 0 to 5% by weight individual ingredient based on the total weight of the composition.

Method of Use

The compositions of the invention are primarily intended for topical application to the skin or hair of a human subject in rinse-off compositions, in order to provide cleansing and optionally other skin or hair benefits such as hydration, nutrition, softness, protection and revitalisation.

The compositions provided by the invention are aqueous compositions, used by massaging them into the skin or hair followed by rinsing with clean water.

The compositions may be applied directly to the skin or hair by hand, or alternatively via an implement such as a natural or synthetic sponge, puff, towel or woven or non-woven cloth. The dilution thickening property of the composition of the invention is especially advantageous when the composition is used with an apertured implement (for example a porous or meshed implement such as a sponge or puff), since the dilution thickening enables the composition to remain in the apertures of the implement for longer, thereby reducing wastage and maximising consumer benefit.

The invention is further illustrated with reference to the following, non-limiting example, in which all percentages are by weight based on total weight unless otherwise specified.

### EXAMPLES

### Examples 1-2

Compositions were prepared having ingredients as shown in Table 1 below.

All ingredients are expressed by weight percent of the total formulation, and as level of active ingredient.

Examples A and B are comparative examples (not according to the invention). Examples 1 and 2 are formulations according to the invention.

TABLE 1

| | Example | Example | Example | Example |
|---|---|---|---|---|
| Ingredient | B | A | 1 | 2 |
| Sodium lauryl | 7 | 7 | 7 | 9 |
| ether sulphate | | | | |
| Cocamide MEA | — | — | 0.9 | 1.15 |
| Cocamidopropyl | 1.4 | 2.0 | 1.4 | 1.7 |
| betaine | | | | |
| Perfume | 0.5 | 0.5 | 0.5 | 0.5 |
| Glycerine | 0.5 | 0.5 | 0.5 | 0.5 |
| Sodium chloride | 0.9 | 1.8 | 1.8 | 1.0 |
| Water | to 100 | To 100 | to 100 | To 100 |

Viscosity Evaluation

The respective viscosity behaviours of the compositions of Examples 1 and 2 were evaluated at various degrees of dilution with water. The results are shown in Tables 2 and 3 respectively below.

TABLE 2

| Dilution* | Viscosity (cps) |
|---|---|
| Neat (undiluted) composition | 6420 |
| 98:2 | 8380 |
| 96:4 | 8880 |
| 94:6 | 9400 |
| 92:8 | 9200 |
| 90:10 | 8800 |
| 88:12 | 8500 |
| 86:14 | 8420 |
| 84:16 | 8360 |

*Measured as the weight ratio of [composition of Example 1]:[water added to the composition to dilute it.]

TABLE 3

| Dilution* | Viscosity (cps) |
|---|---|
| Neat (undiluted) composition | 7600 |
| 98:2 | 8400 |
| 96:4 | 10600 |
| 94:6 | 12500 |
| 92:8 | 13200 |
| 90:10 | 14000 |
| 88:12 | 14200 |
| 86:14 | 14200 |
| 84:16 | 14000 |

*Measured as the weight ratio of [composition of Example 2]:[water added to the composition to dilute it.]

It can be seen for both Examples 1 and 2 that viscosity increases on dilution.

For comparison, the respective viscosity behaviours of the compositions of Examples A and B were evaluated at various degrees of dilution with water. The results are shown in Tables 4 and 5 respectively below.

TABLE 4

| Dilution* | Viscosity (cps) |
|---|---|
| Neat (undiluted) composition | 7360 |
| 98:2 | 6440 |
| 96:4 | 5800 |
| 92:8 | 4840 |
| 88:12 | 3820 |
| 84:16 | 3060 |
| 80:20 | 2340 |

*Measured as the weight ratio of [composition of Example A]:[water added to the composition to dilute it].

TABLE 5

| Dilution* | Viscosity (cps) |
|---|---|
| Neat (undiluted) composition | 6600 |
| 98:2 | 6300 |
| 96:4 | 5940 |
| 92:8 | 5240 |
| 88:12 | 4580 |
| 84:16 | 3980 |

*Measured as the weight ratio of [composition of Example B]:[water added to the composition to dilute it].

It can be seen that the viscosities of the compositions of Examples A and B do not exhibit any increase on dilution.

### Example 3

Compositions were prepared having ingredients as shown in Table 6 below.

All ingredients are expressed by weight percent of the total formulation, and as level of active ingredient.

Examples C is a comparative example (not according to the invention). Example 3 is formulations according to the invention.

TABLE 6

| Ingredient | Example C | Example 3 |
|---|---|---|
| Sodium lauryl ether sulphate | 7 | 7 |
| Cocamide MEA | — | 0.9 |
| Cocamidopropyl betaine | 2.0 | 1.4 |
| Perfume | 0.5 | 0.5 |
| Glycerine | 0.5 | 0.5 |
| Sodium chloride | 2.5 | 0.9 |
| Water | to 100 | to 100 |

Viscosity Evaluation

The respective viscosity behaviour of the composition of Example 3 was evaluated at various degrees of dilution with water. The results are shown in Tables 7 below.

TABLE 7

| Dilution* | Viscosity (cP) |
|---|---|
| 0.3:1 | 8000 |
| 0.8:1 | 50000 |
| 2:1 | 15000 |
| 8:1 | 8000 |
| Neat (undiluted) composition | 7000 |

*measured as the weight ratio of [composition of Example 3]:[water added to the composition to dilute it].

It can be seen that the viscosity of the composition of Example 3 increases on dilution up to a peak (at about 0.8:1 dilution with water), where the viscosity increase is at least 7-fold compared to the neat viscosity of the composition.

For comparison, the viscosity behaviour of the composition of Example C was evaluated at various degrees of dilution with water. The results are shown in Table 8 below.

TABLE 8

| Dilution* | Viscosity (cP) |
|---|---|
| 0.08:1 | 100 |
| 0.2:1 | 200 |
| 0.3:1 | 400 |
| 0.5:1 | 800 |
| 0.7:1 | 2000 |
| Neat (undiluted composition | 8000 |

*measured as the weight ratio of [composition of Example C]:[water added to the composition to dilute it].

It can be seen that the viscosity of the composition of Example C does not exhibit any increase on dilution.

1. A liquid cleansing composition comprising:
(i) 1 to 30% by weight, based on the total weight of the composition, of anionic surfactant;
(ii) up to 5% by weight, based on the total weight of the composition, of amphoteric surfactant;
(iii) up to 2% by weight, based on the total weight of the composition, of electrolyte;
(iv) up to 2% by weight, based on the total weight of the composition, of monoalkanolamide surfactant, and
(v) from 50 to 94.5% by weight, based on the total weight of the composition, of water,
wherein the composition has a viscosity upon dilution which is greater than its viscosity prior to dilution.

2. A composition according to claim 1, in which the anionic surfactant is selected from alkyl ether sulphates of general formula:
R—O—(CH₂—CH₂—O)ₙ—SO⁻₃M⁺
in which R is a straight or branched chain alkyl group having 10 to 18, preferably 12 to 14 carbon atoms, X is a number that represents the average degree of ethoxylation and ranges from 1 to 5, preferably from 2 to 3.5, and M is a alkali metal, ammonium or alkanolammonium cation, preferably sodium, potassium, monoethanolammonium or triethanolammonium, or a mixture thereof.

3. A composition according to claim 1, in which the amphoteric surfactant is selected from alkyl amidopropyl betaines of general formula:
R—CO—NH—(CH₂)₃—N⁺(CH₃)₂—CH₂COO⁻
in which R is a straight or branched chain alkyl group having 10 to 18, preferably 12 to 14 carbon atoms.

4. A composition according to claim 1, in which the electrolyte is sodium chloride.

5. A composition according to claim 1, in which the total amount of electrolyte ranges from 0.3 to 1.8%, more preferably from 0.5 to 1.2%, most preferably from 0.8 to 1.2% by weight based on the total weight of the composition.

6. A composition according to claim 1, in which the monoalkanolamide surfactant is coconut monoethanolamide (cocamide MEA).

7. A composition according to claim 1, in which the total amount of monoalkanolamide surfactant ranges from 0.5 to 2.0%, more preferably from 0.6 to 1.2%, most preferably from 0.8 to 1.0% by weight based on the total weight of the composition.

8. A kit comprising a composition according to claim 1, in combination with an apertured implement for application of the composition to the skin or hair.

9. The use of monoalkanolamide surfactant for lessening the amount of electrolyte required to induce dilution thickening in a liquid cleansing composition.

10. Use according to claim 9, in which the liquid cleansing composition is a composition according to any one of claims 1 to 7.
